(19) United States
(12) Patent Application Publication
(10) Pub. No.: US 2025/0134885 A1
(43) Pub. Date: May 1, 2025

(54) USE OF SHP2 INHIBITORS FOR INHIBITING SENESCENCE

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); ETABLISSEMENT FRANÇAIS DU SANG (EFS), La Plaine Saint Denis (FR); UNIVERSITÉ TOULOUSE III - PAUL SABATIER, Toulouse (FR); CENTRE HOSPITALIER UNIVERSITAIRE DE TOULOUSE, Toulouse (FR); ECOLE NATIONALE VÉTÉRINAIRE DE TOULOUSE, Toulouse (FR)

(72) Inventors: Armelle YART, Toulouse Cedex 4 (FR); Laurène MAZEYRIE, Toulouse (FR); Cédric DRAY, Toulouse (FR); Thomas EDOUARD, Toulouse (FR); Jean-Philippe PRADERE, Toulouse (FR); Philippe VALET, Toulouse (FR)

(21) Appl. No.: 18/860,855

(22) PCT Filed: Mar. 20, 2023
(86) PCT No.: PCT/EP2023/057044

§ 371 Date: Oct. 28, 2024

(30) Foreign Application Priority Data
Mar. 21, 2022 (EP) 22305333.1

(51) Int. Cl. A61K 31/497

(52) U.S. Cl. CPC A61K 31/497

(57) ABSTRACT

According to the WHO, the population aged 60 and over will have doubled by 2050. Unfortunately, aging comes along with an explosion of aging-associated disorders, such as metabolic and cardiovascular diseases, bone and muscle weakening, cognitive dysfunction, that all contribute to the loss of functional capacities, leading to frailty and dependence. Now, the inventors reveal a premature aging phenotype, associating metabolic defects and muscle weakness, in a mouse model of Noonan Syndrome. Both clinical traits are linked to myeloid cells dysfunction and increased senescence, highlighting the role of SHP2 hyperactivation in the onset of aging-associated diseases. Thus the present invention relates to the use of SHP2 inhibitors for inhibiting senescence.

### FIELD OF THE INVENTION

The present invention is in the field of medicine, in particular aging-associated diseases.

### BACKGROUND OF THE INVENTION

According to the WHO, the population aged 60 and over will have doubled by 2050. Unfortunately, aging comes along with an explosion of aging-associated disorders, such as metabolic and cardiovascular diseases, bone and muscle weakening, cognitive dysfunction, that all contribute to the loss of functional capacities, leading to frailty and dependence. The healthy aging is therefore fundamental from a societal and economic point of view, which requires to understand how aging drives these disorders.

Intriguingly, several genetic disorders associate from infancy multiple features that are reminiscent of age-associated diseases, highlighting the single genetic event causing these conditions as a potent target during natural aging. In that context, Noonan Syndrome (NS), a relatively frequent (1/2000 live births) genetic disease associating multiple congenital defects (dysmorphic features, cardiopathies, short stature, developmental delay), seems to be also characterized by a premature aging phenotype. Indeed, patients with NS display musculo-skeletal defects (e.g. decreased bone mass and muscle weakness), predisposition to myeloproliferative disorders (MPD), and metabolic disturbances (e.g. insulin resistance)(1, 2). NS is mainly caused by gain-of-function mutations in PTPN11 gene, encoding the tyrosine phosphatase SHP2. They result in SHP2 hyperactivation, triggering an upregulation of the RAS/MAPK pathway, which has been causally linked to NS pathophysiology (1). Consistent with a premature aging phenotype, activation of oncogenes such as RAS (a SHP2 target) triggers senescence, a cellular stress and damage response that drives aging (8). Moreover, recent data reveal recurrent dysfunctions of myelomomocytic cells (macrophages, myeloid precursors, osteoclasts) in lane with NS clinical signs and the contribution of myeloid cells and an inflammatory component (the so-called inflammaging) in aging-associated diseases is well established (7). However, the contribution of SHP2 hyperactivation may contribute into age-related disorders in general has never been documented.

### SUMMARY OF THE INVENTION

The present invention is defined by the claims. In particular, the present invention relates to the use of SHP2 inhibitors for inhibiting senescence.

### DETAILED DESCRIPTION OF THE INVENTION

The inventors reveal a premature aging phenotype, associating metabolic defects and muscle weakness, in a mouse model of NS. Both clinical traits are linked to myeloid cells dysfunction and increased senescence, highlighting the role of SHP2 hyperactivation in the onset of aging-associated diseases.

Accordingly, the first object of the present invention relates to a method of inhibiting senescence in a subject in need thereof comprising administering to the subject a therapeutically effective amount of a SHP2 inhibitor.

As used herein, the term “subject” refers to an animal, preferably a mammal such as a human or dog, most preferably a human, who is in need of treatment of a disease or condition wherein the inhibition of senescence is beneficial or who is suffering or suspected to suffer from a disease or condition wherein inhibition of senescence is beneficial. Preferably, a subject is at least 30 or at least 40 years old. More preferably, the subject is at least 50 years old.

In some embodiments, the subject is an elderly subject. As used herein, the term “elderly subject” refers to an adult patient sixty-five years of age or older.

In some embodiments, the subject suffers from obesity. In some embodiments, the subject is an elderly subject suffering from obesity, or is at risk of suffering from obesity.

As used herein the term “obesity” refers to a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

As used herein, the term “senescence” has its general meaning in the art and refers to the phenomenon by which normal diploid cells lose the ability to divide under normal conditions. More particularly senescence is the gradual deterioration of functional characteristics in living organisms. The term “senescence” thus refers to either cellular senescence or to senescence of the whole organism.

As used herein, the term “senescent cells” includes cells that are characterized by having an essentially permanent growth arrest. Senescent cells are essentially irresponsive to proliferation-cues. For recognition or detection of senescent cells, molecular markers may be used. Several markers for senescent cells have been developed. The term “senescent cell”, as used herein, includes cells that are characterized by at least one of the following markers, i.e. (i) essentially permanent growth arrest, preferably indicated by a loss of proliferation markers (e.g. cyclin A, MCM-3 and/or PCNA) and insensitivity to growth cues, (ii) senescence-associated β-galactosidase (SA-B-Gal), (iii) p16^{INK4a} activation and/or expression, (iv) p21^{cip1}, preferably p53/p21^{cip1}, activation and/or expression (v) senescence-associated heterochromatin foci (SAHF), (vi) DNA-SCARS (DNA segments with chromatin alterations reinforcing senescence) which often partially co-localize with promyelocytic leukemia protein (PML) nuclear bodies, (vii) the senescence-associated secretory phenotype (SASP), preferably characterized by an elevated (>2-fold) presence of SASP markers, such as IL1, IL6 and/or IL8, as compared to a non-senescent cell or a cell in the direct vicinity of said senescent cell and/or (viii) nuclear export of the non-SASP alarmin HMGB1. The skilled person knows when a cell is considered to be in essentially permanent growth arrest, for example by assessing EdU incorporation and/or Ki67 positivity. The phenomenon of senescence can occur at the end of the proliferative lifespan of normal cells or in normal or tumor cells in response to, for example, chemotherapeutic agents, radiation, DNA damage or other cellular insults. The skilled person can thus distinguish senescent cells from inter alia terminally differentiated cells, which in general do not have the characteristics of senescent cells as described hereinabove. In the context of this paragraph, the term “expression” refers to an increase in gene expression products (RNA) or an increase in protein products as compared to non-senescent cells. The skilled person knows how to measure expression levels of genes and proteins. For example, p16^{INK4a} and p21^{cip1} expression can be measured by immunohistochemistry using antibodies (e.g. Anti-DKN2A/p16INK4a antibody DCS50.1, ab16123, Abcam, UK; and Anti-p21 antibody, ab7960, Abcam, UK). It is described in the art that senescent cells are withdrawn from the cell cycle through independent activity of p53-p21^{cip1} or p 16^{ink4a} each of which is described as individually sufficient for inducing and maintaining the non-proliferative state (Rodier et al., J Cell Biol 192:547-556 (2011)).

In particular, the method of the present invention is particularly suitable for inhibiting a physiological change accompanying senescence. The physiological change includes decrease in physical endurance, fatigue, decrease in energy metabolism, and dysfunction of mitochondria. More specifically, the physiological change includes decrease in physical endurance, fatigue, decrease in energy metabolism, and dysfunction of mitochondria observed in aged people such as people of middle age (i.e., in their 30's or older) or in elderly subjects.

In some embodiments, the method of the present invention is particularly suitable for inhibiting muscle senescence.

As used herein, the term “muscle senescence” refers to weakening of muscle accompanying senescence; for example, muscle dysfunctions (muscle force, muscle physical endurance, and instantaneous muscle power) or muscular atrophy.

More particularly, the SHP2 inhibitors of the present invention are particularly suitable for the treatment of age-associated diseases.

As used herein, the term “age-associated disease” refers to any disease or condition in a mammalian, preferably human, subject wherein the presence of senescent cells, or presence of cellular senescence, or presence of senescence, in a mammalian, preferably human, subject is linked to said disease or condition in said subject. The term “associated” as used herein, refer to a connection between the presence of senescent cells, or presence of cellular senescence, or senescence and said disease or condition. In the aforementioned context, the term “associated” can inter alia refer to the senescent cells or cellular senescence or senescence (i) as the at least partial cause of a disease or condition, (ii) or as a symptom of a disease or disorder.

Examples of age-associated diseases include but are not limited to atherosclerosis, chronic inflammatory diseases such as arthritis or arthrosis, osteoarthritis, diabetes, diabetic ulcers, kyphosis, scoliosis, hepatic insufficiency, cirrhosis, laminopaties, osteoporosis, dementia, (cardio)vascular diseases, obesity, metabolic syndrome, acute myocardial infarction, emphysema, insulin sensitivity, sarcopenia, neurodegenerative diseases such as Alzheimer's, Huntington's or Parkinson's disease, cataracts, anemia, hypertension, fibrosis, age-related macular degeneration, COPD, asthma, renal insufficiency, incontinence, hearing loss such as deafness, vision loss such as blindness, sleeping disturbances, pain such as joint pain or leg pain, imbalance, fear, depression, breathlessness, weight loss, hair loss, muscle loss, loss of bone density, frailty and/or reduced fitness.

According to the present invention, the age-associated disease is not cancer or is not insulin-resistance.

A particular age-related disease wherein the inhibition of senescence is beneficial is a disease or condition associated with or linked to inflammation, preferably chronic inflammation, in a mammalian, preferably human, subject, wherein said inflammation is provided or mediated by senescent cells. Preferably, said senescent cells providing or mediating said inflammation at least partially co-localize in the same organ, more preferably in the same tissue, as the organ, preferably tissue, affected by said disease or condition.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “SHP2” has its general meaning in the art and refers to the protein encoded by the PTPN11 gene. SHP2 is a non-receptor protein tyrosine phosphatase (PTP) with two Src homology-2 (SH2) domains (N-SH2, C-SH2) (Alonso et. al., 2004; Neel et al., 2003). SHP2 is also known as Homo sapiens protein tyrosine phosphatase, non-receptor type 11 (PTPN11).

As used herein, a “SHP2 inhibitor” refers to any compound natural or not which is capable of inhibiting the activity of SHP2, in particular SHP2 phosphatase activity. SHP2 inhibitors are well known in the art. The term encompasses any SHP2 inhibitor that is currently known in the art or that will be identified in the future. The term also encompasses inhibitor of expression. In some embodiments, the SHP2 inhibitor is selective over the other phosphatases including SHP1. By “selective” it is meant that the inhibition of the selected compound is at least 10-fold, preferably 25-fold, more preferably 100-fold, and still preferably 300-fold higher than the inhibition of the other phosphatases. Typical assays are also described in WO2010121212 and WO2015003094. Typically, the SHP2 inhibitor is a small organic molecule.

SHP2 inhibitors are well known in the art (Wu J, Zhang H, Zhao G, Wang R. Allosteric Inhibitors of SHP2: An Updated Patent Review (2015-2020). Curr Med Chem. 2021; 28(19):3825-3842. doi: 10.2174/1568011817666200928114851. PMID: 32988341).

Non-limiting examples of SHP2 inhibitors include NSC-87877 (also known as 8-Hydroxy-7-[(6-sulfo-2-naphthyl)azo]-5-quinolinesulfonic acid), estradiol phosphate, estramustine phosphate, PHPS1, NSC-117199, SP1-112, SP1-112Me (and see Chen, L. et al., 2006 and Chen, L. et al., 2010), tautomycetin analogs (e.g., see Liu, S. et al., 2011), phenylhydrazonopyrazolone sulfate and compounds described in Hellmuth, K. et al., 2008, compounds described in United States Patent Application Publication No. 20120034186 (U.S. Ser. No. 13/274,699) and compounds described in Yu, Z. H. et al. 2011.

Non-limiting example of SHP2 inhibitors also include TNO155 ((3S,4S)-8-(6-amino-5-((2-amino-3-chloropyridin-4-yl)thio)pyrazin-2-yl)-3-methyl-2-oxa-8-azaspiro[4.5]decan-4-amine), RMC-4630 (as described in; WO2018013597; CAS No.: 2172652-48-9), JAB-3068 (CAS No. 2169223-48-5), RLY-1971, ERAS-601, and BBP-398 (CAS No. 2160546-07-4).

In some embodiments, the SHP2 inhibitor for use according to the present invention is selected from compounds described in WO2018013597, WO2010121212, WO2015003094, WO2017100279, and WO2007117699.

In some embodiments, the SHP2 inhibitor for use according to the present invention is SHP009: 6-(4-amino-4-methylpiperidin-1-yl)-3-(2,3-dichlorophenyl)pyrazin-2-amine (Garcia Fortanet J, Chen C H, Chen Y N, Chen Z, Deng Z, Firestone B, Fekkes P, Fodor M, Fortin P D, Fridrich C, Grunenfelder D, Ho S, Kang Z B, Karki R, Kato M, Keen N, LaBonte L R, Larrow J, Lenoir F, Liu G, Liu S, Lombardo F, Majumdar D, Meyer M J, Palermo M, Perez L, Pu M, Ramsey T, Sellers W R, Shultz M D, Stams T, Towler C, Wang P, Williams S L, Zhang J H, LaMarche M J. Allosteric Inhibition of SHP2: Identification of a Potent, Selective, and Orally Efficacious Phosphatase Inhibitor. J Med Chem. 2016 Sep. 8; 59(17):7773-82).

In some embodiments, the SHP2 inhibitor for use according to the present invention is a compound having a formula (IV):

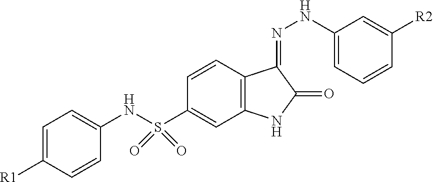

wherein R₁ is selected from the group consisting of F; and wherein R₂ is selected from the group consisting of COOCH₃, and CO₂″▪N⁺H₂(CH₃)(CH₂CHOH)₄CH₂OH).

In some embodiments, the SHP2 inhibitor for use according to the present invention is selected from the group consisting of:

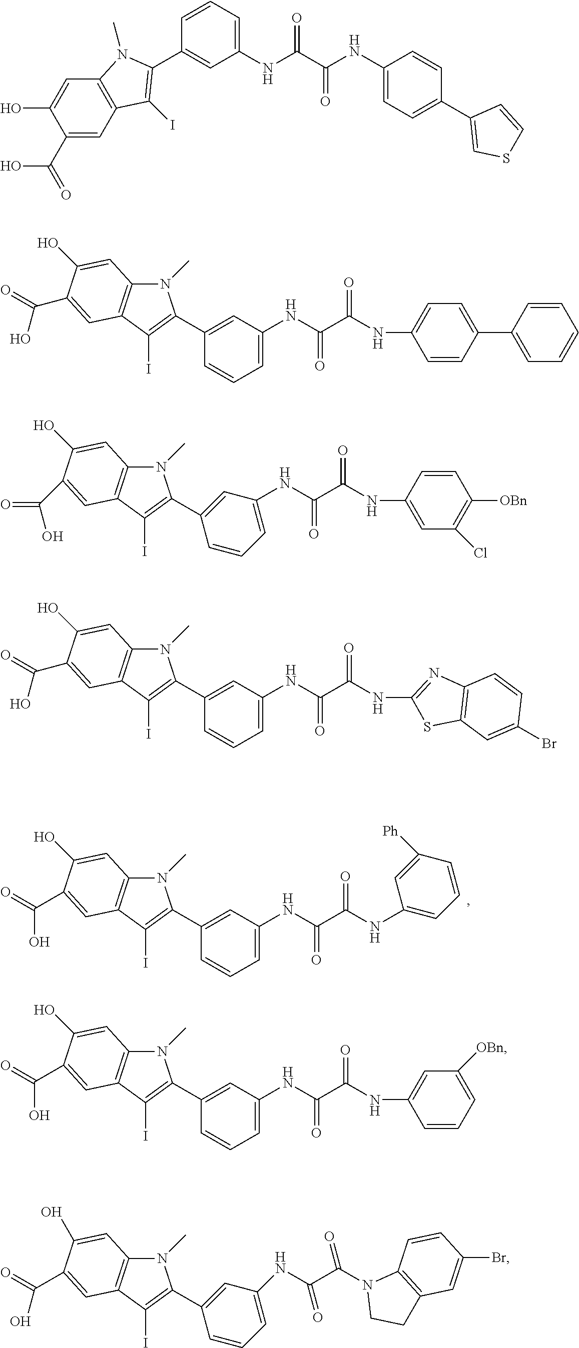

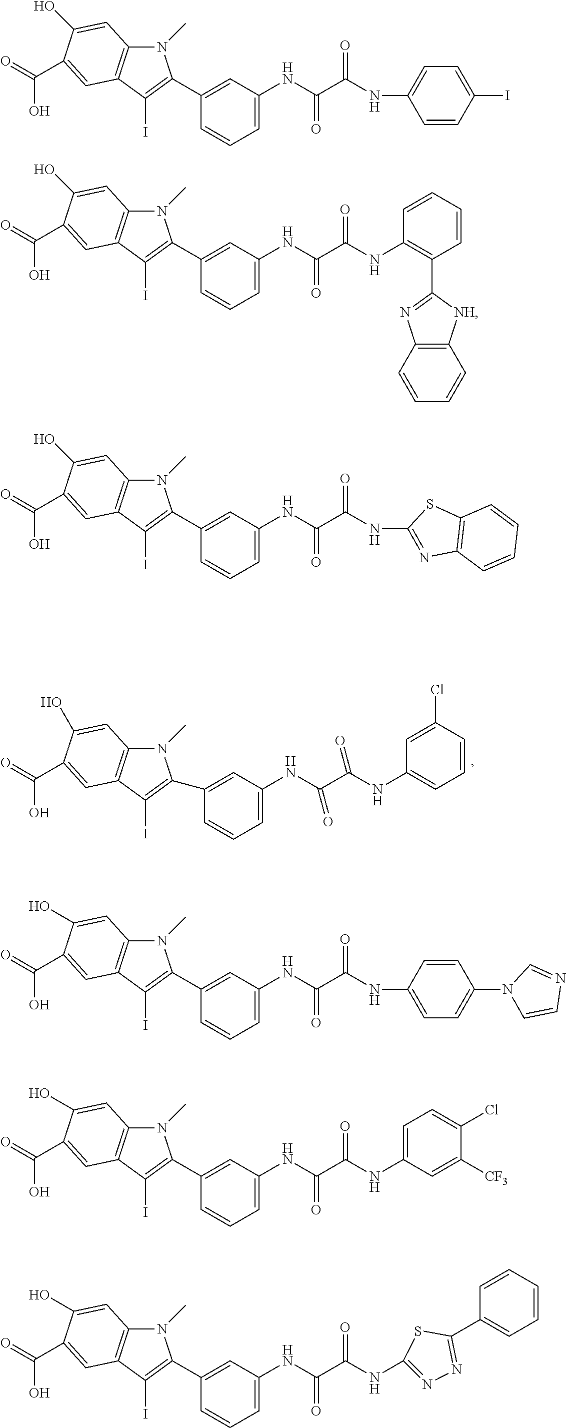

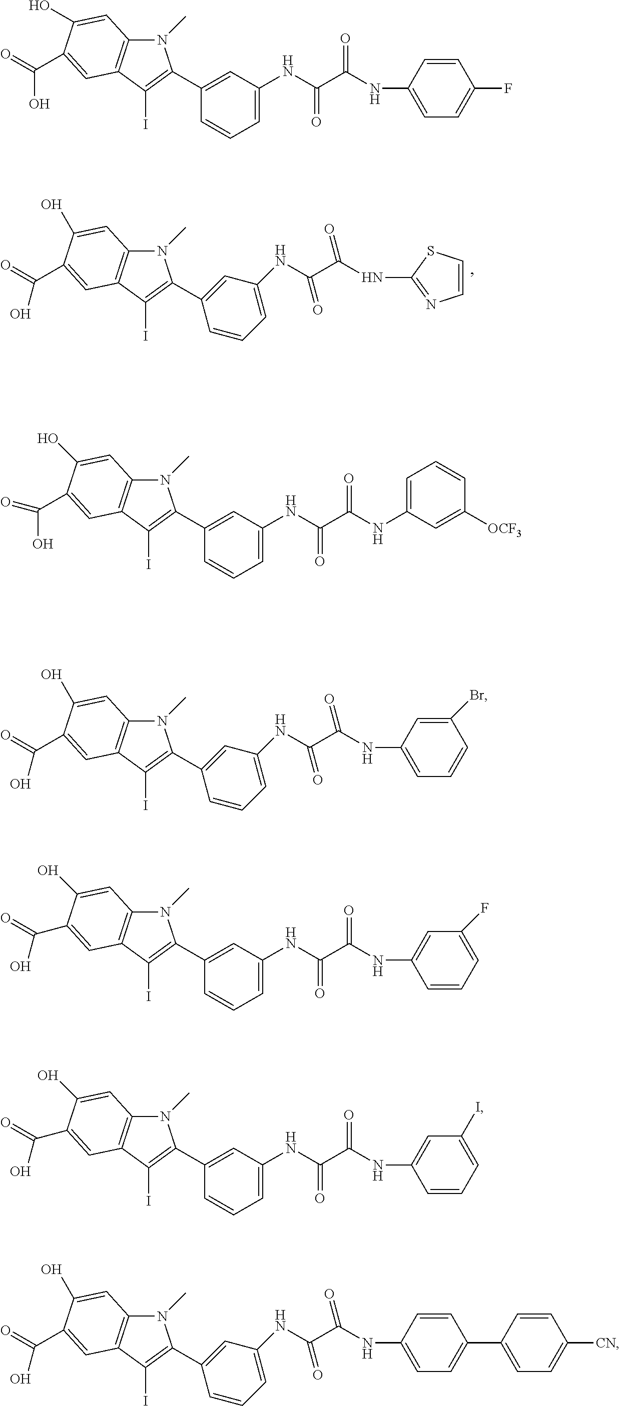

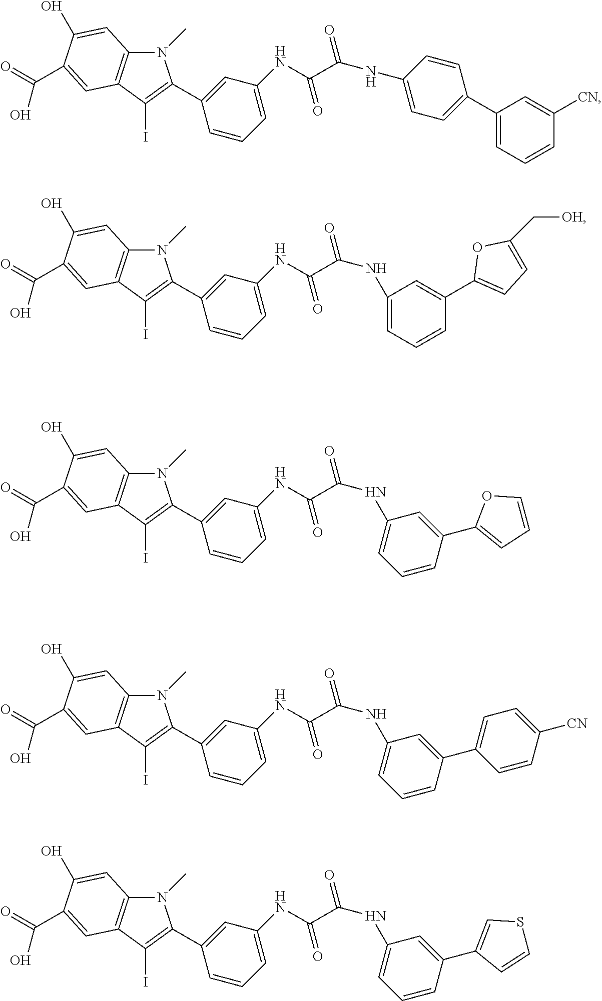 [SMILES: Cn1c(-c2cccc(NC(=O)C(=O)Nc3ccc(-c4cccc(C#N)c4)cc3)c2)c(I)c2cc(C(=O)O)c(O)cc21 ; Cn1c(-c2cccc(NC(=O)C(=O)Nc3cccc(-c4ccc(CO)o4)c3)c2)c(I)c2cc(C(=O)O)c(O)cc21 ; Cn1c(-c2cccc(NC(=O)C(=O)Nc3cccc(-c4ccco4)c3)c2)c(I)c2cc(C(=O)O)c(O)cc21 ; Cn1c(-c2cccc(NC(=O)C(=O)Nc3cccc(-c4ccc(C#N)cc4)c3)c2)c(I)c2cc(C(=O)O)c(O)cc21 ; Cn1c(-c2cccc(NC(=O)C(=O)Nc3cccc(-c4ccsc4)c3)c2)c(I)c2cc(C(=O)O)c(O)cc21]

In some embodiments, the SHP2 inhibitor is an inhibitor of SHP2 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of SHP2 mRNA by binding thereto and thus preventing protein translation or increasing mRNA degradation, thus decreasing the level of SHP2, and thus activity, in a cell. For example, antisense oligonucleotides of at least about 15 bases and complementary to unique regions of the mRNA transcript sequence encoding SHP2 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. SHP2 gene expression can be reduced by contacting a subject or cell with a small double stranded RNA (dsRNA), or a vector or construct causing the production of a small double stranded RNA, such that SHP2 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing SHP2. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus/lentivirus. One can readily employ other vectors not named but known to the art.

In some embodiments, the SHP2 inhibitor is embedded or conjugated to a nanoparticle, so that the SHP2 inhibitor would be preferentially deliver in myeloid cells (e.g. macrophages) that are able to phagocyte said nanoparticle.

As used herein, the term “nanoparticle” encompasses liposomes, polymer micelles, polymer-DNA complexes (polycomplexes), nanospheres, nanofibres, nanotubes, and nanocapsules. All these nanoparticles are known in the art. The surface of such nanoparticles is often modified by PEG brush (PEGylation, i.e. polyethylene glycol (PEG) is attached to the surface of the nanoparticles). In some embodiments, the nanoparticle is a nanocapsule. As used herein, the term “nanocapsules” means vesicular systems in which the drug is confined to a cavity surrounded by a uniquer polymer membrane. In some embodiments, the nanoparticle is a nanosphere. As used herein, the term “nanosphere” means a matrix system in which the drug is physically and uniformly dispersed. In some embodiments, the nanoparticle is a liposome. As used herein, the term “liposome” includes any structure composed of a lipid bilayer that enclose one or more volumes, wherein the volume can be an aqueous compartment. Liposome consist of one, two, three, four, five, six, seven, eight, nine, ten or more lipid bilayers. The term “lipid bilayer” includes, but is not limited to: phospholipid bilayer, bilayer consisting of nonionic surfactants. Liposomes consisting of a phospholipid bilayer can be composed of naturally-derived phospholipids with mixed lipid chains (like e.g. phosphatidylethanolamine), or of pure components like DOPE (dioleolylphosphatidyl-ethanolamine) but are not limited to these components. Liposomes include—but are not limited to—emulsions, foams, micelles, exosomes, vesicles, insoluble monolayers, liquid crystals, phospholipid dispersions, lamellar layers and the like. The term “liposome” also includes so called “stealth liposomes” which consist of water-soluble polymers (e.g. polyethyleneglycol, PEG) attached to the surface of conventional liposomes composed of a lipid mono- or bilayer that enclose a volume (e.g. so called PEGylated liposomes). Following liposome preparation, the liposomes may be sized to achieve a desired size range and relatively narrow distribution of liposome sizes. Methods of coupling inhibitors according to the present invention to liposomes generally involve either covalent cross linking between a liposomal lipid and an inhibitor. In another approach, an inhibitor according to the present invention has been covalently derivatized with a hydrophobic anchor, such as fatty acids, is incorporated into a preformed lipid.

As used herein, the term “therapeutically effective amount” refers to an amount effective, at dosages and for periods of time necessary, to achieve a desired therapeutic result. A therapeutically effective amount of the active agent may vary according to factors such as the disease state, age, sex, and weight of the individual, and the ability of the active agent to elicit a desired response in the individual. A therapeutically effective amount is also one in which any toxic or detrimental effects of drug are outweighed by the therapeutically beneficial effects. The efficient dosages and dosage regimens for the active agent depend on the disease or condition to be treated and may be determined by the persons skilled in the art. A physician having ordinary skill in the art may readily determine and prescribe the effective amount of the pharmaceutical composition required. For example, the physician could start doses of active agent employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the patient's size, the severity of the patient's symptoms, and the particular composition or route of administration selected. An exemplary, non-limiting range for a therapeutically effective amount of a drug of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a drug of the present invention is 0.02-100 mg/kg, such as about 0.02-30 mg/kg, such as about 0.05-10 mg/kg or 0.1-3 mg/kg, for example about 0.5-2 mg/kg.

Typically the active ingredient of the present invention (e.g. SHP2 inhibitor) is combined with pharmaceutically acceptable excipients, and optionally sustained-release matrices, such as biodegradable polymers, to form pharmaceutical compositions.

As used herein, the term “pharmaceutical composition” refers to a composition described herein, or pharmaceutically acceptable salts thereof, with other agents such as carriers and/or excipients. The pharmaceutical compositions as provided herewith typically include a pharmaceutically acceptable carrier.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical-Sciences, Sixteenth Edition, E. W. Martin (Mack Publishing Co., Easton, Pa., 1980) discloses various carriers used in formulating pharmaceutical compositions and known techniques for the preparation thereof. In particular, the pharmaceutically acceptable carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1. RtqPCR was performed on different tissues/cells of WT and NS mice (n=8 per group, 6 months-old), SCAT: subcutaneous adipose tissue, PGAT: perigonadal adipose tissue, BMDM: bone marrow derived macrophages) to quantify p16 expression. Results are expressed as percentage of the WT value, mean+/−SEM. *, p<0.05, **, p<0.01, ***, p<0,005, ****, p<0.001, 1-way ANOVA).

FIG. 2. 10-week-old mice were put under High Fat Diet (HFD) or maintained under normal diet for 10 weeks, then treated i.p. for 4 weeks with SHP099 (25 mg/kg/d) or TNO155 (2.5 mg/kg/d) or vehicle. RtqPCR was performed on liver to quantify p16 expression. Results are expressed as percentage of the WT value, mean+/−SEM. *, p<0.05, **, p<0.01, 1-way ANOVA).

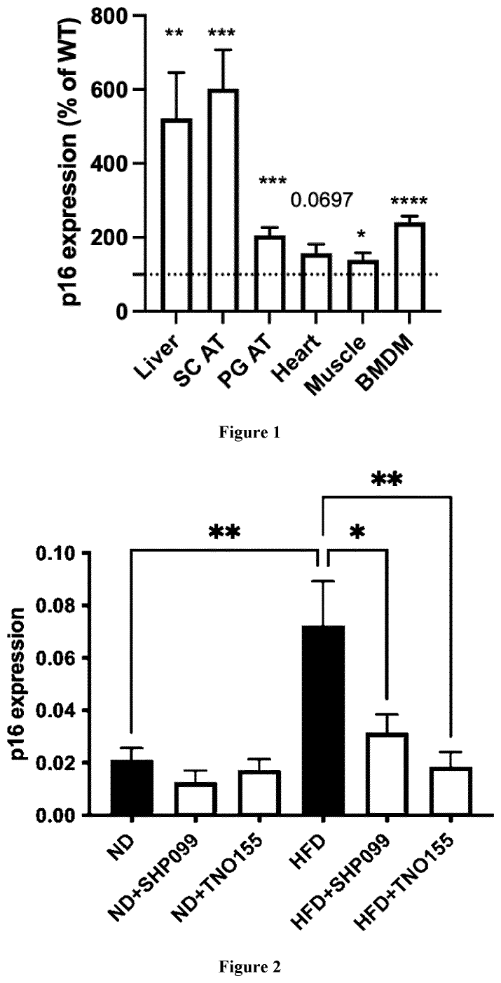

### EXAMPLE

### Methods:

We performed an extensive phenotyping of NS SHP2^{D61G/+} mice phenotype (metabolic imbalance by glucose/insulin resistance tests and analyses on metabolic tissues, muscle frailty by grip test and other functional tests followed by muscle analysis). The role of myeloid cells in these dysfunctions was assessed by bone marrow transplantation (BMT) in lethally irradiated mice. To determine whether and how increased senescence contributed to this phenotype, senescence was measured in different tissues/cells, and genetic reduction of the senescence process was achieved by the mean of p16^{INK4A} gene invalidation in NS mice. Mice fed a high fat diet (HFD), taken as a model of accelerated aging, were treated during four weeks with SHP2 inhibitors, then their glucose tolerance was measured, and correlated with inflammation and senescence markers.

### Results:

NS mice display insulin resistance and reduced muscle mass and strength. This phenotype correlates with an upregulation of inflammatory gene expression in liver, adipose tissue and muscle. Moreover, we find an overexpression of senescence markers, notably p16 in these same tissues.

Repopulation of NS mice with WT bone marrow cells restores insulin sensitivity and increases muscle strength and normalizes p16 expression in the different tissues. Reduction of p16 expression rescues in part the phenotype of NS mice with a recovery of the glucose tolerance. However, it does not alter inflammatory genes expression.

Chronic SHP2 inhibition improves glucose tolerance of HFD mice, with concomitant improvement of inflammation and senescence that are induced by the HFD.

### CONCLUSIONS

These data reveal a premature aging phenotype, associating metabolic defects and muscle weakness, in a mouse model of NS. Both clinical traits are linked to myeloid cells dysfunction and increased senescence, highlighting the role of SHP2 hyperactivation in the onset of aging-associated diseases.

### REFERENCES

Throughout this application, various references describe the state of the art to which this invention pertains. The disclosures of these references are hereby incorporated by reference into the present disclosure.

1. A method of inhibiting senescence in a subject in need thereof comprising administering to the subject a therapeutically effective amount of a SHP2 inhibitor.

2. The method of claim 1 wherein the subject is an elderly subject.

3. The method of claim 1 wherein the subject suffers from obesity, or at risk of obesity.

4. The method of claim 1 wherein the subject is suffering a physiological change accompanying senescence selected from the group consisting of physical endurance, fatigue, decrease in energy metabolism, and dysfunction of mitochondria.

5. The method of claim 1 wherein the subject is suffering from muscle senescence.

6. The method of claim 1 wherein the subject is suffering from an age-associated disease.

7. The method of claim 6 wherein the age-associated disease is selected from the group consisting of atherosclerosis, a chronic inflammatory disease, osteoarthritis, diabetes, diabetic ulcers, kyphosis, scoliosis, hepatic insufficiency, cirrhosis, a laminopathy, osteoporosis, dementia, a (cardio)vascular diseases, obesity, metabolic syndrome, acute myocardial infarction, emphysema, insulin sensitivity, sarcopenia, a neurodegenerative diseases, cataracts, anemia, hypertension, fibrosis, age-related macular degeneration, COPD, asthma, renal insufficiency, incontinence, hearing loss, vision loss, a sleeping disturbance, pain, imbalance, fear, depression, breathlessness, weight loss, hair loss, muscle loss, loss of bone density, and frailty and/or reduced fitness.

8. The method of claim 6 wherein the age-associated disease is associated with or linked to inflammation.

9. The method of claim 1 wherein the SHP2 inhibitor is embedded in or conjugated to a nanoparticle, so that the SHP2 inhibitor is delivered to myeloid cells that are able to phagocytize said nanoparticle.

10. The method of claim 7 wherein the chronic inflammatory diseases is arthritis or arthrosis.

11. The method of claim 7 wherein the neurodegenerative disease is Alzheimer's, Huntington's or Parkinson's disease.

12. The method of claim 7 wherein the hearing loss is deafness.

13. The method of claim 7 wherein the vision loss is blindness.

14. The method of claim 7 wherein the pain is joint pain or leg pain.

15. The method of claim 8, wherein the inflammation is chronic inflammation.
